(19) United States
(12) Patent Application Publication
(10) Pub. No.: US 2018/0301208 A1
(43) Pub. Date: Oct. 18, 2018

(54) BLOOD TREATMENT DEVICE

(71) Applicant: FRESENIUS MEDICAL CARE DEUTSCHLAND GMBH, Bad Homburg (DE)

(72) Inventors: Peter ZEYHER, Darmstadt (DE); Kim FRITSCH, Trebur (DE)

(73) Assignee: FRESENIUS MEDICAL CARE DEUTSCHLAND GMBH, Bad Homburg (DE)

(21) Appl. No.: 15/752,135

(22) PCT Filed: Aug. 9, 2016
(86) PCT No.: PCT/EP2016/001363

§ 371 Date: Feb. 12, 2018

(30) Foreign Application Priority Data
Aug. 11, 2015 (DE) 10 2015 010 430.6

(51) Int. Cl. G16H 10/65; A61M 1/16; G06K 19/04; G06K 7/00; A61M 1/36

(52) U.S. Cl. CPC G16H 10/65; A61M 1/1601; G06K 19/044; A61M 1/287; A61M 1/3621; A61M 2205/52; A61M 1/1692; G06K 7/0021

(57) ABSTRACT

The present invention relates to a blood treatment device having at least one reading and/or writing apparatus which is configured to read information from a card and/or to write information to a card, and having at least one receiver into which the card can be introduced and from which the card can be removed, wherein the receiver is arranged such that information can be read from the card and/or information can be written to the card by means of the reading and/or writing apparatus in the state of the card introduced into the receiver, wherein the receiver is not horizontal, but rather extends slanted upwardly with respect to the horizontal in the direction of introduction of the card.

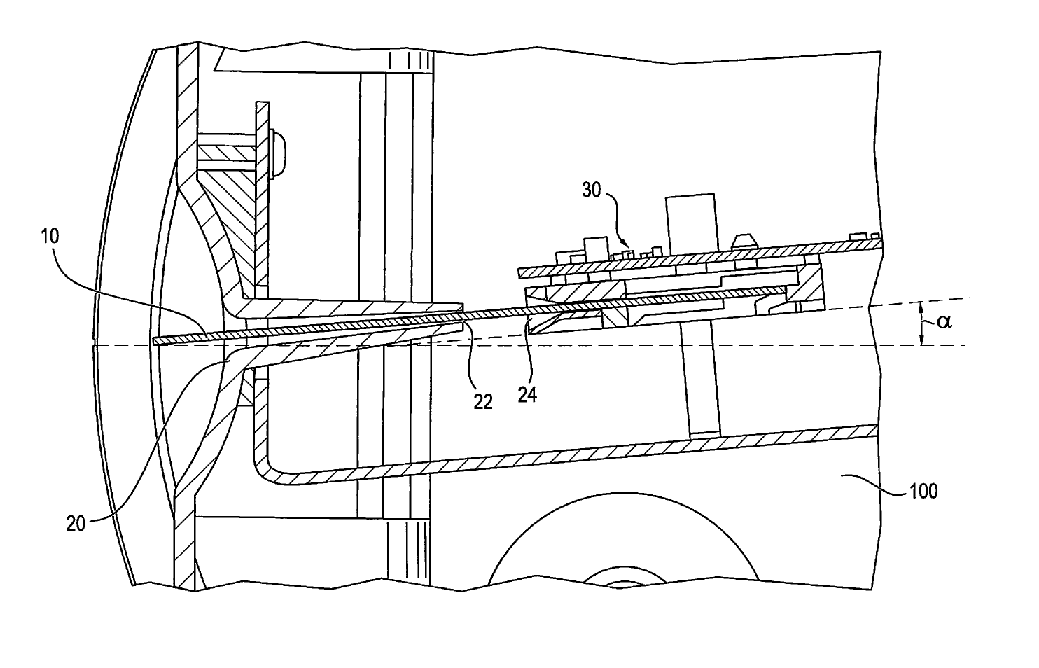

The present invention relates to a blood treatment device having at least one reading and/or writing apparatus which is configured to read information from a card and/or to write information to a card, and having at least one receiver into which the card can be introduced and from which the card can be removed, wherein the receiver is arranged such that information can be read from the card and/or information can be written to the card by means of the reading and/or writing apparatus in the state of the card introduced into the receiver.

Blood treatment devices are known from the prior art, in particular in the form of peritoneal dialysis machines, in which the solution bags containing the dialyzate are arranged on or above the actual machine or machine housing.

There is the risk in this respect that liquid exiting the bags drips onto the machine housing, and in particular onto a chip card located in the receiver, such as a patient card, and then penetrates into the machine. The penetrating liquid can cause damage to the electronics, for example to the reading and/or writing apparatus in the device, or can contaminate it so that the legibility or the writability of the card is impaired accordingly.

It is the object of the present invention to further develop a blood treatment device of the initially named kind such that the probability for the penetration of liquid into the receiver is reduced with respect to known devices.

This object is achieved by a blood treatment device having the features of claim 1.

Provision is accordingly made that the receiver is not horizontal, but is rather aligned sectionally or entirely slanted upwardly with respect to the horizontal in the direction of introduction of the card.

It is achieved with comparatively simple means by this inclination of the receiver that the penetration of liquid into the receiver is avoided. If liquid should impact on the inserted chip card or enter into the region of the introduction opening of the receiver, said liquid will run off downwardly or outwardly and not into the receiver.

The term “card” is to be understood generally and is not restricted to card-like or plate-like articles. Pin-like elements or elements of other shapes are also covered by the term “card”. The term “card” is representative for an element of any desired geometrical shape from which data can be read and/or onto which data can be written.

Such cards are typically patient cards on which patient data or data of the treatment at the blood treatment device are recorded.

The card is preferably a chip card, i.e. a card having an integrated circuit which e.g. has a memory or also a microprocessor.

In a preferred embodiment of the invention it is a plastic card such as is also known, for example, from ATM cards. As stated, the invention is, however, not restricted to a card shape in a narrower sense, but rather relates to all other geometrical shapes.

The reading and/or writing apparatus is configured to read information from the chip card and/or to write information to the chip card or to another card. This information can, for example, be the just performed or just running blood treatment.

In a further embodiment, the receiver has at least one introduction opening, preferably a slit, with the reading and/or writing apparatus being located in a region which is spaced apart from the introduction opening.

The reading and/or writing apparatus is preferably located in the end region of the receiver spaced apart from the introduction opening.

It is thereby particularly reliably ensured that liquid dripping onto the chip card does not move into the region of the card chip or into the region of the reading and/or writing apparatus.

In a preferred embodiment of the invention, the blood treatment device is a dialysis machine.

It is particularly preferred if the blood treatment device is a peritoneal dialysis machine.

In a peritoneal dialysis machine, a machine housing with the total machine control or machine regulation is typically present, with provision preferably being made that the receiver for the card is located in this housing. Receivers or suspension apparatus for solution bags filled with dialysis solution are typically located above this housing.

If liquid from these bags moves into the region of the receiver, it is prevented by the slanted arrangement of the receiver that the liquid runs into the receiver. It is rather achieved that the liquid runs off or drips off to the outside.

The receiver preferably has at least one introduction opening, preferably at least one introduction slit, for the card, with no separate means being provided in the region of the introduction openings to avoid the penetration of liquid into the introduction opening. This allows a particularly simple design in which, for example, projections, etc. can be dispensed with which lead the liquid away or keep it away from the receiver. The penetration of liquid into the receiver is preferably only prevented by its slanted design.

There is preferably also no cover for the introduction opening since—as stated—the penetration of liquid into the receiver is prevented by its slanted arrangement.

The angle between the receiver and the horizontal is preferably in the region of <45°, preferably in the region of <20°. A particularly advantageous angle is at 10° to 20°, and for example at 15°.

The receiver can extend slanted upwardly with respect to the horizontal over its total length extending in the introduction direction of the card or also only over a part section of this length.

It is preferred if the receiver is slanted in total and only has a single gradient. Different embodiments are, however, also covered. If the inclination of the receiver along the introduction direction changes, this requires a correspondingly flexible design of the card.

The receiver can be one-part or also multi-part, with a gap being able to be present between the parts of the receiver in the last case.

The shape of the receiver is arbitrary. It can be a hollow space which is substantially of rectangular shape in accordance with the contour of the card. With differently shaped cards, correspondingly other shapes of the receiver are also conceivable such as elliptical, triangular, polygonal, etc.

Further details and advantages of the invention will be explained in more detail with reference to an embodiment shown in the drawing.

The only FIGURE shows a section through the housing of a peritoneal dialysis machine in the region of the card receiver.

Reference numeral 100 marks the chassis or the housing of a peritoneal dialysis machine in which the electronics for carrying out the blood treatment are located. Attachment possibilities, not shown, for solution bags containing the dialyzate required for the treatment are located above the housing 100.

A patient card is marked by reference numeral 10 and is a chip card which is received in the receiver 22, 24. The receiver 22, 24 is formed by a hollow space which approximately corresponds in its dimensions to the outer dimensions of the card 10.

Reference numeral 20 marks the introduction slit for the card 10.

As can be seen from the FIGURE, the receiver 22, 24 is multi-part, with the plurality of parts 22, 24 being spaced apart from one another. The card 10 first passes through the part 22 of the receiver in the insertion direction and then the part 24 of the receiver, with this part having a funnel-like introduction opening to ensure that the card also enters reliably into the part 24 of the receiver on the introduction.

Reference numeral 30 marks a reading and/or writing apparatus which is configured to read the data of the patient card 10 or to read the data from its chip and/or to write information to it.

As can be seen from the illustration, the receiver is configured in accordance with the embodiment such that it has a depth which is below the length of the chip card 10 in the introduction direction so that the chip card projects outwardly beyond the introduction slit 20 in the introduced state.

In order now in particular to avoid that liquid dripping onto this projecting part of the card 10 runs into the receiver 22, 24 and in particular into the region of the receiver 24 in which the reading and/or writing apparatus is arranged, the receiver 22, 24 is inclined in total by the angle α with respect to the horizontal. The receiver 22, 24 is inclined upwardly in the introduction direction of the card 10 so that the liquid ripping onto the projecting part of the card 10 does not run into the receiver, but rather drips off from the card outside the receiver.

The angle α is, for example, an angle of 15°, with this angle only being of an exemplary nature.

There is neither a projection nor a flap, which keeps off liquid dripping down, in the region of the introduction slit 20. This is also not necessary since the inclination of the receiver prevents the running of liquid into the receiver 22, 24. The comparatively small inclination of the receiver with respect to the horizontal allows a substantially horizontal introduction direction of the card which is then aligned in accordance with the inclination of the receiver, i.e. faces or is introduced slanted upwardly.

1. A blood treatment device having at least one reading and/or writing apparatus which is configured to read information from a card and/or to write information to a card, and having at least one receiver into which the card can be introduced and from which the card can be removed, wherein the receiver is arranged such that information can be read from the card and/or information can be written to the card by means of the reading and/or writing apparatus in the state of the card introduced into the receiver, characterized in that the receiver is not horizontal, but rather extends slanted upwardly with respect to the horizontal in the introduction direction of the card.

2. A blood treatment device in accordance with claim 1, characterized in that the card is a chip card; and in that the reading and/or writing apparatus is configured to read information from the chip card and/or to write information to the chip card.

3. A blood treatment device in accordance with claim 1, characterized in that the receiver has at least one introduction opening for the card; and in that the reading and/or writing apparatus is located in the end region of the receiver spaced apart from the introduction opening.

4. A blood treatment device in accordance with claim 1, characterized in that the blood treatment device is a dialysis machine.

5. A blood treatment device in accordance with claim 1, characterized in that the blood treatment device is a peritoneal dialysis machine.

6. A blood treatment device in accordance with claim 1, characterized in that the receiver is located in a housing; and in that one or more solution bags are located above the housing; or in that one or more receivers for solution bags are arranged above the housing.

7. A blood treatment device in accordance with claim 1, characterized in that the receiver has at least one introduction opening for the card; and in that no means for avoiding the penetration of liquid into the introduction opening are present in the region of the introduction opening.

8. A blood treatment device in accordance with claim 1, characterized in that the receiver has at least one introduction opening for the card; and in that no cover for the introduction opening is present.

9. A blood treatment device in accordance with claim 1, characterized in that the angle between the receiver and the horizontal is in the range of <45°, preferably in the range of <30°, and particularly preferably in the range of <20°.

10. A blood treatment device in accordance with claim 1, characterized in that the receiver extends slanted upwardly with respect to the horizontal over it total length extending in the introduction direction of the card or only over a part section of this length.
